(19) United States
(12) Patent Application Publication
(10) Pub. No.: US 2021/0396738 A1
(43) Pub. Date: Dec. 23, 2021

(54) 3D HUMAN CANCER MODEL-BASED COMBINATORIAL DRUG DEVELOPMENT METHOD

(71) Applicant: PreComb Therapeutics AG, Hombrechtikon (CH)

(72) Inventors: Jens Michael KELM, Uster (CH); Peter STEINER, Altendorf (CH)

(21) Appl. No.: 17/292,046

(22) PCT Filed: Nov. 20, 2019
(86) PCT No.: PCT/EP2019/081977

§ 371 Date: May 7, 2021

(30) Foreign Application Priority Data
Nov. 20, 2018 (GB) 1818885.4

(51) Int. Cl. G01N 33/50; G06F 16/21

(52) U.S. Cl. CPC G01N 33/5082; G06F 16/211; G01N 33/5011; G01N 33/5014

(57) ABSTRACT

The present invention relates to a method of characterizing a composition comprising two or more active drug compounds, the method comprising the steps of: a) a composition selection screen (CSS), in which screen a candidate composition comprising two or more active drug compounds is tested against a 3D microtissue derived from one or more cell line, and b) a composition validation screen (CVS), in which screen the candidate composition of step b) is tested against a 3D microtissue derived from a primary patient sample.

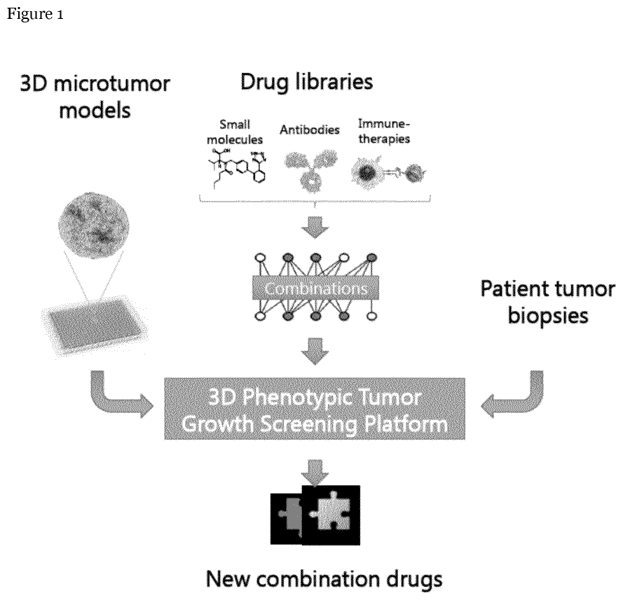

The present invention relates to a method of characterizing a composition comprising two or more active drug compounds, the method comprising the steps of: a) a composition selection screen (CSS), in which screen a candidate composition comprising two or more active drug compounds is tested against a 3D microtissue derived from one or more cell line, and b) a composition validation screen (CVS), in which screen the candidate composition of step b) is tested against a 3D microtissue derived from a primary patient sample (FIG. 1).

### FIELD OF THE INVENTION

The present invention relates to the screening of drug combinations for therapeutic purposes. Generally, drugs for therapeutic purposes are screened for efficacy in a conventional screening system, where drugs from a library are tested in a suitable cell-based assay. Usually, the viability of the cells and/or the cytotoxicity of the candidate drug is investigated.

This approach can often be done in high throughput, but incurs a high risk that drugs identified in such approach as promising will disappoint in the subsequent clinical testing. Furthermore, it has turned out that in specific fields of indication, drug combinations are increasingly used, e.g., to avoid the development of resistances, or to exploit synergistic effects.

So far, almost no systematic approaches have been disclosed to screen potential drug combinations at early stage. Conventionally, drugs are combined empirically, by medical practitioners, and tested in patients. However, a systematic approach to really investigate the combinatorial effects of such drug combination is missing. This means that a huge potential of promising drug combination exists but never sees the patient, for lack of systematic investigation.

Also, the predictability of early stage experiments to the future in vivo situation needs to be improved, in order to reduce the risk of failure of drug combinations that have turned out promising in the pre-clinic.

Further, there is a need to determine novel drug combinations faster, more efficiently and with sustainable therapeutic efficacy.

WO 2013/050962A1 relates to a tumor microenvironment platform for culturing tumor tissue comprising drugs, a method of predicting the response of a tumor to drugs and a method of screening or developing anti-cancer agents.

WO 2017/081260A1 relates to the use of a three-dimensional spheroid for the screening of potentially therapeutic agents, wherein said screening is a high-throughput screening of a library of potentially therapeutic agents.

US 2016/040132A1 refers to bioprinted three-dimensional pancreatic tumor tissues and a method of identifying therapeutic agents therefor.

None of said documents mentions the necessity of studying the combinatorial effect of two or more drugs, and the advantages of such newly identified drug combinations.

### EMBODIMENTS OF THE INVENTION

These and further objects are met with methods and means according to the independent claims of the present invention. The dependent claims are related to preferred embodiments. It is yet to be understood that value ranges delimited by numerical values are to be understood to include the said delimiting values.

### SUMMARY OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limiting values.

Further, it is to be noted that documents or contents which are incorporated by reference herein are mainly meant to provide enabling disclosure, so as to avoid lengthiness of the text.

According to one embodiment of the invention, a method of characterizing a physiological effect of a composition comprising two or more active drug compounds is provided. The method comprises the steps of:

b) a composition selection screen (CSS), in which screen a 3D microtissue derived from one or more cell lines is exposed to said composition comprising two or more active drug compounds, and/or

c) a composition validation screen (CVS), in which screen 3D microtissue derived from a primary patient sample is exposed to the composition of step b), so as to characterize a physiological effect of said composition on the 3D microtissue.

It is important to understand that steps b) and c) can be carried out in the same location or in different locations. Step b) can for example be carried out in a laboratory that has access to cell lines, while step b) can be carried out in a laboratory that is closes to clinical site, and has for that reason access to primary patient samples.

In one embodiment, the protocols carried out in steps b) and c), including parameters such as for example
microtissue size, microtissue weight, or cell number in microtissues,
dosing of the composition comprising two or more active drug compounds
timing of the exposure to the composition comprising two or more active drug compounds
composition of the culture media, and/or
culturing conditions, like pH, temperature, O₂/CO₂ flow, etc. are identical, or almost identical, or essentially identical, as technically feasible.

This allows a high degree of reproducibility and comparability of results obtained in step b) (CSS) and c) (CVS), as well as a standardization between the two steps. Further, the 3D model enables to work within the same format, independent of the cell source. By contrast, in 2D models it is sometimes not easy to have tumor cells grow in culture. Further, this approach allows a high degree of automatization in both steps.

Generally, some regulatory authorities demand that, when a combination of two or more standard of care drugs is applied for, an added patient value of such combination is demonstrated. The above approach can deliver data supporting such added value already at a very early stage of development, by providing two independent screening stages early in the process under similar experimental conditions.

As used herein the terms “microtissue exposed to a composition” and “composition tested against a microtissue” mean essentially the same. A “composition comprising two or more active drug compounds” will also be called “drug combination” or “combination of drugs” herein.

As used herein the terms “active drug compound” relates to the ingredient in a pharmaceutic that is biologically active. The term is used interchangeably with the term “active pharmaceutical ingredient (API)”.

A “3D microtissue derived from one or more cell lines” is a 3D multi-cellular 3D microtissue comprising at least one cell line selected from the group consisting of
immortalized cell lines,
cancer cell lines,
cancer cells from a tumor xenograft, and/or,
cancer cells from a patient-derived xenograft.

A “3D microtissue derived from a primary patient sample” is a 3D multi-cellular 3D microtissue as obtained from dissociated tumors, as e.g. obtained from tumor biopsies, tumors obtained from surgery, organ donations, and the like.

The cells used for such 3D microtissue can be fresh, e.g., taken directly after biopsy or surgery, or can be cryopreserved cells.

Generally, in order to produce 50.000 3D microtissues, a total of 12.5 mio-25 mio cancer cells are required. While large tumors surgically removed from a patient often comprise such large numbers of cells, patient tumor material obtained by a biopsy will usually have less cells.

For this reason, in step b), a 3D microtissue derived from one or more cell lines is used. This provides the advantage that such cell material is available in large quantities, hence allowing the acquisition of a large number of data points. This is necessary, inter alia, for screening through large libraries of drug combinations, at different concentrations.

In step c) such 3D microtissue derived from a primary patient tumor sample is used. This material is usually not available in large quantities.

However, the concept of 3D microtissues allows to generate a sufficient number of micromodels of tumors from a patient sample. These micromodels faithfully reflect the physiology and genetics of a real tumor, instead of cancer cell lines, which very often differ in physiology and genetics from a real tumor.

Hence, this approach allows already to screen drug combinations for a specific patient cohort or patient stratum, and is thus an important step to provide a clinical or therapeutic response to the demands posed by increasing patient stratification.

3D microtissues are preferably cultured in a particular vessel, e.g. a microreaction vessel or a well of a microtiter plate. Exposing the 3D microtissues to a drug or drug combination means, for example, that said anti-cancer agent means is added to that vessel, e.g., by means of a suitable pipette, pipetting robot or dispenser.

3D microtissues provide a more representative, organotypic model for assessment of tumor growth. They contain layers of cells that exhibit more in vivo-like size- and gradient-dependent proliferation and viability profiles. Further, 3D microtissues allow to recapitulate the native tumor microenvironment. For these reasons, cells in a 3D microtissue behave more physiologically than cells in 2-dimensional cell culture, because they can better establish intercellular communication pathways as well as extracellular matrices. Furthermore, 3D microtissues better reflect physicochemical conditions in a true tissue or organ, because it better simulates diffusion gradients of both gases, like oxygen, and chemical agents. Further, they better simulate penetration barriers for larger components.

Still another advantage is that a 3D microtissue derived from cell lines also allows to tailor a cancer model which faithfully reflects a true tumor, by combining drug resistant and sensitive cells within the same tissue to specifically monitor drug effects on either cell population.

Further, compared to 2-dimensional cell cultures, 3D microtissues have a significantly higher lifetime. While 2-dimensional cell cultures comprising non-immortalized primary cells have an assay lifetime of 3-7 days, 3D microtissues have a lifetime of up to 30 days or even longer, making them suitable for long term investigations of the 3D microtissues to drug exposure—just like in an in vivo situation, where a tumor responds on drug administration's given as singular bolus over an extended period of time.

Yet another advantage is that, while 2-dimensional cell cultures allow mere quantification of resistant colonies (endpoint measurement), 3D microtissues according to the invention allow to observe the short- and longtime kinetic of a tissue response on drug exposure.

Furthermore, optionally, in either the CSS step and/or in the CVS step, control experiments are performed in which a reference drug is tested against one or more 3D microtissues derived from one or more cell lines and/or one or more 3D microtissue derived from a primary patient sample.

Such reference drug is preferably the standard of care for the disease against which the drug combination that is screened for should be active.

As an alternative, such reference drug is preferably the standard of care (SOC) for the disease represented, or modeled, by the 3D microtissue derived from one or more cell lines and/or the 3D microtissue derived from a primary patient tumor sample.

The following table shows typical SOC treatments for pancreatic and non-small cell lung cancer:

| Pancreatic Cancer | Non-small cell lung cancer |
|---|---|
| Capecitabine (Xeloda) | Carboplat in (Paraplatin) or cisplatin |
| | (Platinol) |
| Erlotinib (Tarceva) | Docetaxel (Docefrez, Taxotere) |
| Fluorouracil (5-FU) | Gemcitabine (Gemzar) |
| Gemcitabine (Gemzar) | Nab-paclitaxel (Abraxane) |
| Irinotecan (Camptosar) | Paclitaxel (Taxol) |
| Leucovorin (Wellcovorin) | Pemetrexed (Alimta) |
| Nab-paclitaxel (Abraxane) | Vinorelbine (Navelbine) |

In such way, an added value (efficacy) of a novel drug combination compared to current standard of care drugs can be demonstrated very early on.

In one embodiment, each drug combination is tested against a plurality of 3D microtissues derived from one or more cell line, and/or against a plurality of 3D microtissues derived from a primary patient sample.

In one embodiment, each drug combination is tested against ≥2, ≥3, ≥4, ≥5, ≥6, ≥7, ≥8, ≥9, ≥10, ≥15, ≥20, ≥25, or ≥30 3D microtissues derived from one or more cell lines.

In one embodiment, each drug combination is tested against ≥2, ≥3, ≥4, ≥5, ≥6, ≥7, ≥8, ≥9, ≥10, ≥15, ≥20, ≥25, or ≥30 3D microtissues derived from a primary patient sample.

In one approach, each drug combination is tested against between ≥2 and ≤10 (preferably against between ≥3 and ≤5) 3D microtissues derived from one or more cell lines, and against between ≥5 and ≤50 (preferably against between ≥10 and ≤30) 3D microtissues derived from a primary patient sample.

This allows a very detailed patient specific efficacy stratification of the different drug combinations, and hence increases the likelihood that a drug combination that demonstrates promising results in the CVS step in a given 3D microtissue derived from a primary patient sample will also be successful in the clinic, in particular in a patient cohort that corresponds to the respective 3D microtissue.

In one embodiment, this approach is combined with molecular profiling of the respective tissues, as discussed below, to further stratify the respective 3D microtissues derived from primary patient samples and match them with respective patient cohorts.

According to one embodiment of the invention, at least one parameter representing the characterized physiological effect is generated or determined in the method. Such parameter is for example size or viability.

According to one embodiment of the invention, the method further comprises a range finding step a) (RFS), in which step a plurality of 3D microtissues derived from one or more cell lines are exposed to different concentrations of each compound of the candidate composition, so as to determine suitable concentration ranges of the compounds.

This range finding step can have a particular importance, in particular when drug compounds are chosen which have already been used in the clinic. It might be tempting, for such drug compounds, to use the dosage that has been established in the clinic, however, such dosage may turn out difficult to down- or upscale to the 3D microtissue environment. Generally, clinical dosages are indicated as mg/kg or mg/m2. It is obvious that such dosages, which have been established for systematic administration in a human patient, cannot simply be extrapolated to an in vitro setting with 3D microtissues.

Again, in this step, 3D microtissues derived from one or more cell lines are used. This provides the advantage that such cell material is available in large quantities, hence allowing the acquisition of a large number of data points. This is necessary, inter alia, for screening through large libraries of drugs, at different concentrations.

Hence, the dosage range finding step provides useful information which allows the subsequent screening steps to operate with physiologically relevant drug concentrations which may give indications for the relative doses in pre-clinical assessment.

Another advantage is that the range finding results in combination with the single treatment data from the CCS stage can be used as quality control parameter to check reproducibility. This helps to benchmark drug combinations can be benchmarked against single drug effects. Single drug effects should match growth profiles of the range step of respective concentrations.

According to one embodiment of the invention, the method further comprises a step of obtaining a molecular profile of at least one of:

a) the 3D microtissues derived from one or more cell line, and/or

b) the 3D microtissues derived from a primary patient sample,

According to one embodiment of the invention, the step of molecular profiling is used to detect genomic aberrations, and/or mRNA or protein expression levels.

Such step of molecular profiling can comprise at least one of the steps shown in the following table 1:

TABLE 1
molecular profiling options

| Method | Purpose |
|---|---|
| Immunohistochemistry (IHC) | determines level of protein expression |
| Chromogenic/Fluorescence in | detects gene deletions, amplifications, |
| situ Hybridization (CISH/FISH) | translocations and fusions |
| Next-Generation | detects DNA mutations, copy number |
| Sequencing (NGS) | variations and gene fusions across the |
| | genome |
| Sanger Sequencing | examines strands of DNA to identify |
| | mutations by analyzing long contiguous |
| | sequencing reads |
| Pyro Sequencing (PyroSeq) | detects and quantifies mutations, |
| | methylation, etc. through sequencing by |
| | synthesis |
| Quantitative Polymerase | amplifies and quantifies a targeted DNA |
| Chain Reaction (qPCR) | molecule |
| Fragment Analysis | detects changes in DNA or RNA to |
| (FA/Frag Analysis) | indicate the presence or absence of |
| | genetic marker |
| GeneChip array based | measures expression levels of large |
| analysis | numbers of genes simultaneously or |
| | serves to genotype multiple regions of a |
| | genome |

In such way, the molecular profile of the tissue under investigation can be correlated to the physiological response thereof to exposure to the drug (RFS) or drug combination (CSS, CVS).

According to one embodiment of the invention, the parameter representing the characterized physiological effect is determined over time in at least one of step a), b) and/or c).

This allows to, to inter alia mimic the in vivo situation where a tumor is exposed to changing serum titers of a drug or drug combination, and may or may not develop resistances, or to capture other kinetic effects. Further, disease progression in response to the drug exposure can be captured.

According to one embodiment of the invention, the parameter representing the characterized physiological effect is the size of the 3D microtissue.

According to one embodiment of the invention, the size, relative size and/or the relative size change over time is determined in at least one of step a), b) and/or c).

These approaches mimic the clinical characterization of tumors, which is subject to the so-called RECIST criteria.

Response evaluation criteria in solid tumors (RECIST) is a set of published rules that define when tumors in cancer patients improve (“respond”), stay the same (“stabilize”), or worsen (“progress”) during treatment.

The RECIST specification establishes a minimum size for measurable lesions, limits the number of lesions to follow and standardizes unidimensional measures. Patients with measurable disease at baseline are included in protocols where objective tumor response is the primary endpoint, measured as size changes over time.

According to the respective guidelines, such size is preferably measured with CT and MRI (optical slice thickness of 10 mm or less). The guidelines emphasize that tumor markers alone cannot be used to assess response, while cytology and histology can be used in order to do so.

Hence, the approach according to the embodiment outlined above mimics, on an in vitro basis, the clinical characterization of a tumor's response to a given treatment. This is unique, and greatly enhances the predictive effect of the method according to this embodiment.

This means that drug combinations identified with the method according to this embodiment as potentially efficacious are less prone to fail in subsequent preclinical or clinical evaluation. In other words: the chance that drug combinations identified with the method according to this embodiment will prove successful in subsequent preclinical or clinical evaluation increases significantly as compared to methods according to the prior art.

According to one embodiment of the invention, the size determination of the 3D microtissue refers to at least one parameter selected from the group consisting of:
diameter
perimeter
volume
area of an optical cross section

The size can thus either be a parameter that has directly been measured, or a parameter which has been calculated on the basis of such measurements.

Preferably, the size determination of the 3-dimensional cell culture or tissue is carried out by means of an imaging device.

One example of such imaging device is the Cell3iMager manufactured by SCREEN Holding Co., LTD, Japan. It allows analysis of spheroids by scanning multi-well plates in a bright-field. It computes estimated values based on spheroid size and density, together with spheroid number and area in each well. With easy and efficient operability, its vibration-free design protects cells from damage. An excellent application is also available to determine spheroid proliferation over time and to measure the granular distribution in 3D culture.

According to another preferred embodiment the determination of the effect of the anti-cancer agent exposure on the 3-dimensional cell culture or tissue is determined by
kinetic measurements of cell proliferation and/or
cell viability, and/or endpoint measurements of cell proliferation and/or cell viability.

The term “kinetic measurement” (also called “real time measurement”) is related to those types of measurement in which a given parameter is monitored continuously or frequently during the exposure of the 3-dimensional cell culture or tissue to the at least one anti-cancer agent, including times of interruption of the exposure. Preferably, said kinetic measurement of cell proliferation and/or cell viability is a size determination, e.g., diameter, volume or area of an optical cross section.

Conventional cell-based drug screening assays work on the basis of 2D cell cultures, where

(i) cells are plated to a plating density of 2,500-20,000 cells per well of a multi-well plate

(ii) plated cells are exposed to the drugs to be tested, and

(iii) the cellular response on the exposure is measured by means of a cell viability assay or a cytotoxicity assay (i.e., how many cells have been killed after a defined treatment time, usually 48-72 h).

Hence, these assays rely on a one-point analysis of the drug impact on the cells. In contrast thereto, the method according to the above embodiment analyses the drug impact on the cells over time. This approach does more faithfully reflect the in vivo Situation, where a tumor is exposed to a drug bolus and then responds on it over time.

Further, the parameters that are analyzed in these assays (viability or cytotoxicity) do not comply with the parameters that are referred to by the RECIST guidelines (=size). Hence, the method according to the above embodiment is more compliant with the RECIST criteria, and hence more translatable with regard to the clinical impact of a drug that has successfully been tested.

In other words: A drug that has shown to be active in the method according to the above embodiment is more likely to be clinically successful than a drug that has stood a conventional cell-based drug screening assay.

According to one embodiment of the invention, the size, relative size and/or the relative size change is determined in at least one of step a), b) and/or c) over a period of >1 and <30 days.

Preferably, the size, relative size and/or the relative size change is determined over a period of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 days.

According to one embodiment of the invention, the 3D microtissue is exposed to the active drug compound (step a) or to the composition comprising two or more active drug compounds (steps b and c) only once, by application of a defined bolus.

Said defined bolus reflects one dosage as determined in the range finding step a) as discussed above.

According to one embodiment of the invention, the 3D microtissue is exposed to the active drug compound (step a) or to the composition comprising two or more active drug compounds (steps b and c) two or more times, by application of a defined bolus each.

In this embodiment, a clinical administration scheme can be reproduced, where a patient receives a drug or drug combination several times, with pauses in between. In such clinical scenarios, tumors sometimes develop adaptive resistances to the given therapy. The embodiment discussed above is suitable to reveal whether a given tumor type would be likely to develop an adaptive resistance against a given drug combination.

Very often, chemotherapy or antibody therapy is given to a patient in an intermittent dosage regimen. In such treatment regimen, the serum concentration of the drug is a function of the amount of the dosage and the administration interval.

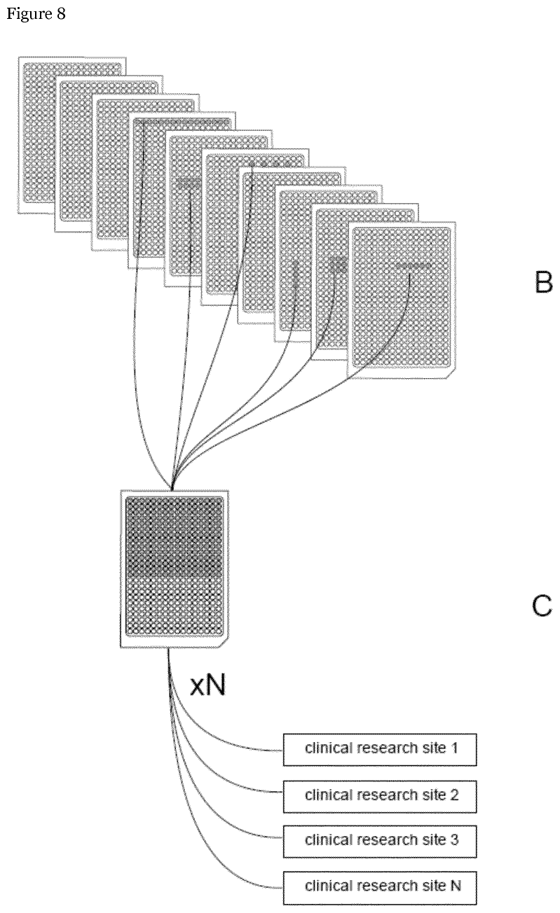

See for example FIG. 1B of Cartron et al., 2007 (reproduced as FIG. 8 herein) which shows that, in an interval dosage regimen, between the different administrations, the serum titer of a drug quickly decreases again.

According to one embodiment of the invention, the composition comprising two or more active drug compounds is removed after the microtissue was exposed thereto for a given period of time.

Such removal may take place in such way that the culture solution comprising the composition is replaced by a suitable culture solution devoid of the composition. Such replacement can take place in one step, or in several incremental steps, to better reproduce the gradual decrease of drug titer in a patient, between the different drug administrations.

According to one embodiment, the 3D microtissue is exposed to the active drug compound (step a) or to the composition comprising two or more active drug compounds (steps b and c) for a duration of <8h.

In one non-limiting example, in any RFS, CSS and/or CVS, the tissue is exposed at day 1 to the composition comprising two or more active drug compounds for 6 hours. Then, the drug combination is replaced by drug free culture medium stepwise over further 6 hours, or in one step. The tissue is then incubated in drug free culture medium, and at day 3, the tissue is exposed again to the composition comprising two or more active drug compounds, again for 6 hours.

The determination of the physiological parameter takes place routinely, in a high throughput system, from day 1 one, and continues for example until day 30, to capture long term effects of the exposure.

In another non-limiting example, the tissue is exposed at day 1 to the composition comprising two or more active drug compounds for 6 hours. Then, the drug combination is replaced by drug free culture medium stepwise over further 6 hours, or in one step. The tissue is then incubated in drug free culture medium, and at day 3, the tissue is exposed again to the composition comprising two or more active drug compounds, again for 6 hours. The determination of the physiological parameter takes place routinely, in a high throughput system, from day 1 one, and continues for example until day 30, to capture long term effects of the exposure.

The following tables 2 and 3 show some preferred protocols that can be used in the context of the present invention. Note that, contrary to what is shown in the tables, the size determination can be carried out continuously, in hourly or daily intervals, or at specifically selected time points.

TABLE 2
Protocol 1

| | | | |
|---|---|---|---|
| Day <0 | Production and | Production and | Production and |
| | maturation of | maturation of | maturation of |
| | microtissue, initial | microtissue, initial | microtissue, initial |
| | size determination | size determination | size determination |
| Day 0 | 8 hrs exposure, then | 48 hrs exposure | 8 hrs exposure, then |
| | drug combination | | drug combination |
| | replaced by culture | | replaced by culture |
| | medium in one step | | medium |
| | or gradually | | |
| Day 2 | 8 hrs exposure, then | Drug combination | 8 hrs exposure, then |
| | drug combination | replaced by culture | drug combination |
| | replaced by culture | medium over the | replaced by culture |
| | medium in one step | next 14 days | medium |
| | or gradually | | |
| Days | Continuous size | Continuous size | 8 hrs exposure |
| 2-14 | determination every | determination every | every |
| | second day | second day | second day, then |
| | | | drug combination |
| | | | replaced by culture |
| | | | medium |
| Day 14 | Size determination | Size determination | Size determination |
| | and subsequent | and subsequent | and subsequent |
| | analysis (e.g. | analysis (e.g. | analysis (e.g. |
| | molecular profiling) | molecular profiling) | molecular profiling) |
| | End of experiment | End of experiment | End of experiment |
| optional | Whole experiment | Whole experiment | Whole experiment |
| | can be looped 2-3x | can be looped 2-3x | can be looped 2-3x |
| | to assess | to assess | to assess |
| | adaptive resistance | adaptive resistance | adaptive resistance |
| | response | response | response |

TABLE 3
Protocol 2

| | | |
|---|---|---|
| Day <0 | Production and maturation | Production and maturation |
| | of microtissue, initial size | of microtissue, initial size |
| | determination | determination |
| Day 0 | 8 hrs exposure, then drug | 48 hrs exposure, then drug |
| | combination replaced by | combination replaced by |
| | culture medium in one step | culture medium in one step |
| | or gradually | or gradually |
| Days 2-13 | Continuous size | Continuous size |
| | determination every second | determination every second |
| | day | day |
| Day 14 | 8 hrs exposure, then drug | 48 hrs exposure, then drug |
| | combination replaced by | combination replaced by |
| | culture medium in one step | culture medium in one step |
| | or gradually | or gradually |
| Days 15-30 | Continuous size | Continuous size |
| | determination every second | determination every second |
| | day | day |
| Day 30 | Size determination and | Size determination and |
| | subsequent analysis (e.g. | subsequent analysis (e.g. |
| | molecular profiling) | molecular profiling) |
| | End of experiment | End of experiment |

According to a further embodiment of the invention, a further step of composition toxicity testing (CTS) is provided, in which step

(i) a microtissue representing connective tissue is exposed to the composition of step b), and/or

(ii) a tissue specific microtissue is exposed to the composition of step c), so as to characterize a physiological effect of said composition on said microtissue.

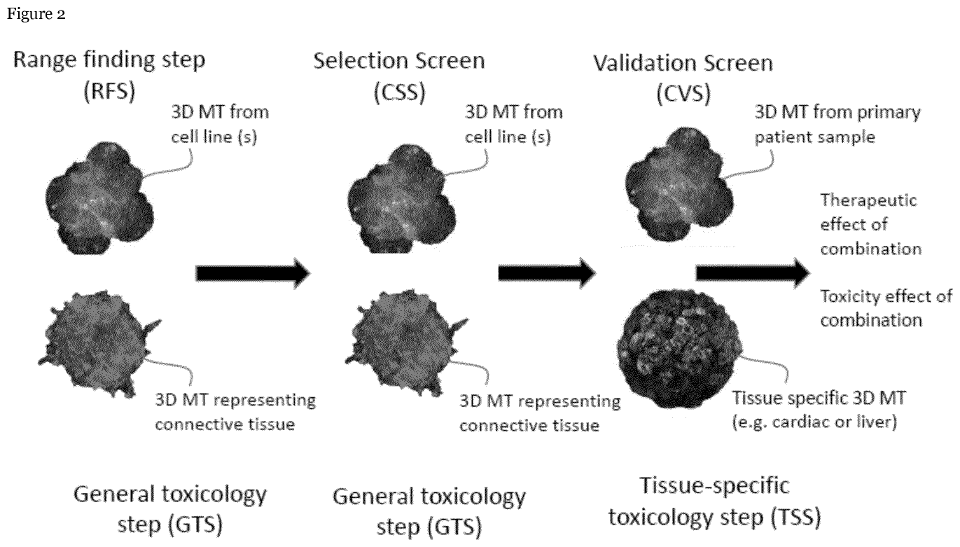

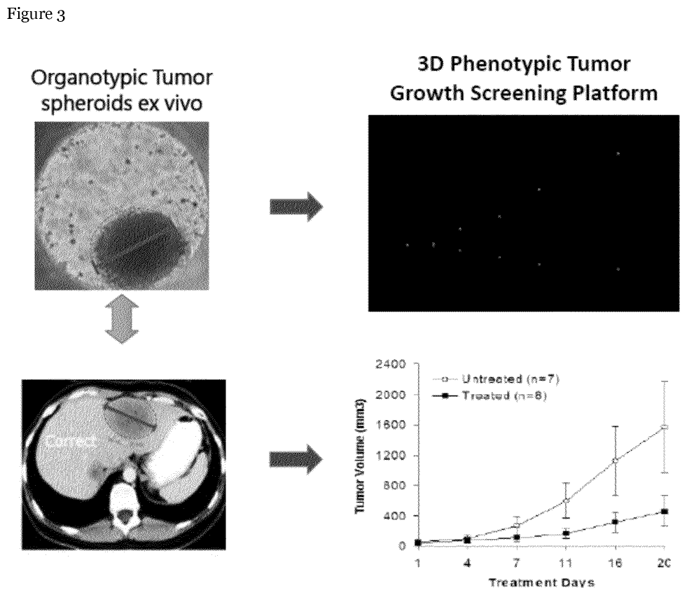

Different types of microtissues can be used. In particular, it is important to understand that in this step, the (i) microtissue representing connective tissue and/or the (ii) tissue specific microtissue do not necessarily have to be a 3D microtissue, different to what FIG. 2 may suggest. In one embodiment, however, the (i) microtissue representing connective tissue and/or the (ii) tissue specific microtissue is a 3D microtissue.

As used herein, a “microtissue representing connective tissue” is a microtissue that comprises connective tissue cells, like, e.g., fibroblasts.

Methods to generate such microtissues are for example disclosed in Kelm et al., J Biotechnol. 2005 Aug. 4; 118(2):213-29, the content of which is incorporated herein by reference.

As used herein, a “tissue specific microtissue” is a microtissue that has a cell composition that is representative for toxicity sensitive tissues, like e.g.
primary hepatocytes, hepatocyte cell lines or stem cell derived hepatocytes (also called “liver model” herein), or
primary cardiomyocytes cells, cardiomyocyte cell lines, or stem cell derived cardiomyocytes (also called “cardiac model” herein)

Methods to generate such liver models are for example disclosed in
Tuschl et al, Chem Biol Interact. 2009 Sep. 14; 181(0:124-37 (Sandwich culture)
Bokhari et al, J Anat. 2007 October; 211(4):567-76. (Scaffold-based liver models)
Beckwitt et al, Exp Cell Res. 2018 Feb. 1; 363(1): 15-25 (Liver chips)
Proctor et al., D. Arch Toxicol. 2017 August; 91(8):2849-2863 (3D Liver microtissues) The content of these documents is incorporated herein by reference.

Methods to generate such cardiac models are for example disclosed in
Sidorov et al., Acta Biomater. 2017 Jan. 15; 48:68-78 (Heart on a chip)
Giacomelli et al., Development. 2017 Mar. 15; 144(6): 1008-1017 (3D Microtissues)
Hansen et al., T. Circ Res. 2010 Jul. 9; 107(1):35-44 (Engineered heart tissue) The content of these documents is incorporated herein by reference.

The microtissue representing connective tissue and the tissue specific microtissue serve as reference models to capture non-proliferation specific cytotoxicity, as well as general cytotoxicity, like e.g. hepatotoxicity and cardiac toxicity.

Next to assessing combinatorial effects to better kill a tumor, this approach allows to gather information to which extent the combination leads to increased toxicity in vitro. This allows not only to demonstrate combinatorial effects but directly whether there any severe changes in the toxicological profiles prior entering the pre-clinical development.

Moreover, drug combinations may allow to reduce single drug concentrations, which might lead to less toxicity effects. Capturing this effect can become an important value driver.

Hence, while in the CSS and CVS, the physiological effect that is characterized can be qualified as therapeutic efficacy, the physiological effect that is characterized in the CTS is a toxicity effect.

In one embodiment, the protocols carried out in the CTS are identical, or almost identical, as technically feasible, to those used in steps b) (CSS) and c) (CVS), including Parameters such as for example
microtissue size, microtissue weight, or cell number in microtissues,
dosing of the composition comprising two or more active drug compounds
timing of the exposure to the composition comprising two or more active drug compounds
composition of the culture media, and/or
culturing conditions, like pH, temperature, O₂/CO₂ flow, etc.

In one embodiment, the characterized physiological effect in the CTS is the size of the microtissue representing connective tissue and/or the tissue specific microtissue.

The size can be is actual size, relative size and/or relative size change over time, and the size determination can refer to at least one parameter selected from the group consisting of:
diameter
perimeter
volume, and/or
area of an optical cross section.

In another embodiment, the characterized physiological effect in the CTS is viability and/or cytotoxicity. Examples for such assays are for example disclosed in Kijanska & Kelm J. In vitro 3D Spheroids and Microtissues: ATP-based Cell Viability and Toxicity Assays. Assay Guidance Manual [Internet]. Bethesda (Md.): Eli Lilly & Company and the National Center for Advancing Translational Sciences; 2004-2016 Jan. 21. The content of this document is incorporated by reference herein.

Generally, in long term studies, viability assays and cytotoxicity assay are much more resource intensive, i.e., they require more sample material. This is because, usually, in a viability assay or a cytotoxicity assay, an IC50 is determined, meaning the drug concentration upon which 50% of the sample material is dead. Hence, in a protocol where the cell or tissue response is to be measured over time (e.g., to capture the effects of elongated exposure, or the capture the development of potential resistances, each measurement would require its own microtissue (meaning that, if for example 5 measurements would be carried out over time, five tissues would necessary). This is contrary to an approach where e.g. size changes of a microtissue are measured as a response to drug exposure (which leaves the tissues alive), so (meaning that, if for example five measurements would be carried out over time, this can be done with only one tissue).

Now as discussed above, the 3D microtissues used in step c) (CVS) are derived from a primary patient tumor sample. This material is usually not available in large quantities.

For this reason, a responsible use of resources is necessary, which also makes the size determination as discussed above advantageous.

In contrast thereto, the material used for the production of the microtissue representing connective tissue and/or the tissue specific microtissue as used in the CTS can, under some circumstances, be available in larger quantities, suggesting that the higher demands as regards resources that are associated with a viability assay or cytotoxicity assay can be tolerated.

On the other hand, in the CSS and CVS, the physiological effect that is characterized is therapeutic efficacy, which is advantageously measured by size determination over time, as discussed above, to inter alia mimic the in vivo situation where a tumor is exposed to changing serum titers of a drug or drug combination, and may or may not develop resistances, or to capture other kinetic effects. Further, disease progression in response to the drug exposure can be captured.

In contrast thereto, a toxicity effect does not necessarily have to be measured over time, because the kinetics of the toxicity response are not always necessary to be determined. So, in one embodiment, in the CTS, the physiological effect of the tissue exposure to the drug combination is only characterized once, preferably by a viability assay or cytotoxicity assay.

In one embodiment, at least one of these microtissues is also subjected to a step of obtaining a molecular profile, as discussed above. In such way, the molecular profile of the tissue under investigation can be correlated to the physiological response thereof to exposure to the drug combination (CTS).

By simultaneously or not simultaneously assessing in vitro toxicity of the screened combinations it is possible to generate an in vitro-based risk-assessment and compare the toxicology profile against standard of care drugs. In addition to increased efficacy of combination drugs, it is hence possible to reduce drug concentrations, and, in such way, reduce side effects, without compromising efficacy. This can only be assessed by an early incorporation of toxicity assessment.

The (i) microtissue representing connective tissue, and/or (ii) the tissue specific microtissue, can be taken from the same patient as the primary patient sample. This ensures a high genetic match between the two microtissues, so as to warrant a high cross-referenceability of results obtained with the respective different 3D microtissues.

In another embodiment, a library of (i) 3D microtissues representing connective tissue, and/or (ii) tissue specific microtissues can be prepared from different test person or patients, which library then serves as a reference library for 3D microtissues representing connective tissue and/or tissue specific microtissues for testing cytotoxicity.

This embodiment can be useful in case it is impossible to obtain, from the patient from which the tumor sample has been obtained, also tissue samples for creating the tissue specific microtissues—e.g., when the patient is severely ill.

In such case the different members of said library can be characterized, molecularly, and can be selected for corresponding testing according to their molecular profiles.

As exemplarily shown in FIG. 3, the tests related to (i) the microtissue representing connective tissue and/or (ii) the tissue specific microtissue can be carried out simultaneously with steps b), c) and or a), or can be carried out a different time and/or place, e.g., in order to create a reference database comprising toxicity data (non-proliferation specific cytotoxicity, as well as general cytotoxicity) of given drug combinations.

In this context, cardiac and hepatic are the most frequent adverse event targets of anti-cancer chemotherapeutic drugs.

Hepatotoxicity implies chemical-driven liver damage. Drug-induced liver injury is a cause of acute and chronic liver disease. The liver plays a central role in transforming and clearing chemicals and is susceptible to the toxicity from these agents. Certain medicinal agents, when taken in overdoses and sometimes even when introduced within therapeutic ranges, may injure the organ. Other chemical agents, such as those used in laboratories and industries, natural chemicals (e.g., microcystins) and herbal remedies can also induce hepatotoxicity. Chemicals that cause liver injury are called hepatotoxins.

More than 900 drugs have been implicated in causing liver injury and it is the most common reason for a drug to be withdrawn from the market. Hepatotoxicity and drug-induced liver injury also account for a substantial number of compound failures, highlighting the need for drug screening assays, such as stem cell-derived hepatocyte-like cells, that are capable of detecting toxicity early in the drug development process. Chemicals often cause subclinical injury to the liver, which manifests only as abnormal liver enzyme tests. Drug-induced liver injury is responsible for 5% of all hospital admissions and 50% of all acute liver failures.

Cardiotoxicity is the occurrence of heart electrophysiology dysfunction or muscle damage. The heart becomes weaker and is not as efficient in pumping and therefore circulating blood.

Cardiotoxicity may be caused by chemotherapy treatment, complications from anorexia nervosa, adverse effects of heavy metals intake, or an incorrectly administered drug such as bupivacaine.

Hence, this embodiment delivers, at a preclinical state, organ specific toxicity assessments. One the basis of the above, the two screening steps deliver, optionally, the following information:

CSS (Combinatorial selection screen)
Combination Efficacy
Permeability/Bioavailability
Unspecific Toxicity

CVS (Combinatorial validation screen)
Tumor-specific concentration
Combination Efficacy
Patient stratification
Organ-specific toxicity

According to one embodiment of the invention, at least one 3D microtissue has been produced in a hanging drop culture System or a low adhesion well culture System.

One example of such hanging drop culture System is the GravityPLUS™ hanging drop system manufactured by InSphero AG, Schlieren, CH. This system allows scaffold-free re-aggregation of single cells into functional 3D microtissues, because it avoids the Provision of liquid/solid interfaces to which cells can adhere.

One example of such low adhesion well culture system Costar® ultra-low attachment multiple well plate manufactured by Coming® or the GravityTRAP™ plate from InSphero. These plates comprise non-adhesively coated wells which allow the formation of 3-dimensional cell cultures or tissues by avoiding adherence of the cells to the solid interface.

According to one embodiment of the invention, the molecular profile of at least one 3D microtissue is correlated with at least one parameter which characterizes the physiological effect as obtained in the composition selection screen (CSS) or the composition validation screen (CVS) of said 3D microtissue.

Further, such correlation between molecular profile and the at least one parameter that characterizes the physiological response can also be done regarding the microtissues used in the range finding step/RFS) and/or the step of composition toxicity testing (CTS).

According to one embodiment, the molecular profile of at least one 3D microtissue is correlated with at least one parameter which characterizes the physiological effect as obtained in the composition selection screen (CSS) or the composition validation screen (CVS) of said 3D microtissue.

In addition, or independently, the molecular profile of at least (i) one microtissue representing connective tissue and/or (ii) one tissue specific microtissue can be correlated with at least one parameter which characterizes the toxicity effect as obtained in the step of composition toxicity testing (CTS).

According to one other embodiment, the method further comprises the step of creating or feeding a database with datasets comprising at least the following entries each:

a) at least one molecular profile of at least one 3D microtissue, and

b) at least one parameter which characterizes the physiological effect as obtained in the composition selection screen (CSS) or the composition validation screen (CVS) of said 3D microtissue.

In addition, or independently, a database can be created, or fed, with datasets comprising at least the following entries each:

a) at least one molecular profile of at least (i) one microtissue representing connective tissue and/or (ii) one tissue specific microtissue, and

b) at least one parameter which characterizes the toxicity effect as obtained in the step of composition toxicity testing (CTS).

According to one embodiment of the invention, the method further comprises the step of creating or feeding a database with datasets comprising at least the following entries each:

a) at least one molecular profile of at least one 3D microtissue, and

b) at least one parameter which characterizes the physiological effect as obtained in the composition selection screen (CSS) or the composition validation screen (CVS) of said 3D microtissue.

Further, into such data base also molecular profiles and parameters that characterizes the physiological response regarding the microtissues used in the range finding step (RFS) and/or the step of composition toxicity testing (CTS) can be entered.

According to another aspect of the invention, a method of screening a plurality of compositions comprising two or more active drug compounds, preferably from one or more libraries, is provided, which method comprises

(i) the application of two or more methods as set forth in any of the aforementioned description, with a different composition of two or more active drug compounds in each individual method, and/or

(ii) several pairs of steps b), c) and optionally a).

According to one embodiment of the invention, the compositions comprising two or more active drug compounds differ from one another by

a) the composition of active drug compounds, or

b) the dosages or concentrations of the active drug compounds in the composition

According to one embodiment of the invention, the method further comprises at least one step selected from the group consisting of a) synthesizing the active drug compounds that are comprised in the compositions,

b) composing the compositions comprising two or more active drag compounds, and/or

c) creating a library comprising active drug compounds that are comprised in the compositions and/or compositions comprising two or more active drug compounds.

According to another aspect of the invention, the method of creating a database is provided, in which method the molecular profile of at least one 3D microtissue is correlated with the result of a composition selection screen (CSS) or a composition validation screen (CVS) of said 3D

According to one embodiment, the molecular profile of at least one 3D microtissue is correlated with at least one parameter which characterizes the physiological effect as obtained in the composition selection screen (CSS) and/or the composition validation screen (CVS) of said 3D microtissue.

In addition, or independently, the molecular profile of at least (i) one microtissue representing connective tissue and/or (ii) one tissue specific microtissue can be correlated with at least one parameter which characterizes the toxicity effect as obtained in the step of composition toxicity testing (CTS).

Likewise, in addition, or independently, a database can be created, or fed, with datasets comprising at least the following entries each:

a) at least one molecular profile of at least (i) one microtissue representing connective tissue and/or (ii) one tissue specific microtissue, and

b) at least one parameter which characterizes the toxicity effect as obtained in the step of composition toxicity testing (CTS).

### Figures and Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent Claims does not indicate that a combination of these measures cannot be used to advantage. Any reference signs in the Claims should not be construed as limiting the scope.

### FIGURES

FIG. 1 shows aspects of the concept of the present invention.

FIG. 2 shows an overview of the different method Steps of the present invention. Note that the steps shown in italics are optional. Note also that the screen for toxicity effects can be done

a) simultaneously or not simultaneously with the RFS, CVS and/or CSS, and/or

b) with (i) 3D microtissues representing connective tissue and/or (ii) tissue specific microtissues taken from the same patient as the primary patient sample, or from a library of (i) 3D microtissues representing connective tissue and/or (ii) tissue specific microtissues prepared from different test person or patients. Note that the screen for toxicity effects can be done a) simultaneously or not simultaneously with the RFS, CVS and/or CSS, and/or

b) with (i) 3D microtissues representing connective tissue and/or (ii) tissue specific microtissues taken from the same patient as the primary patient sample, or from a library of (i) 3D microtissues representing connective tissue and/or (ii) tissue specific microtissues prepared from different test person or patients.

FIG. 3 shows that one aspect of the present invention is capable to mimic the clinical characterization of tumors, which is subject to the so-called RECIST criteria. The growth curve of tumor microtissues is shown for pancreatic tumor microtissues either untreated or treated with Gemcitabine (500 mg/m²). It demonstrates that the same kind of data can be generated with an in vitro assay as pre-clinical animal data and ultimately in vivo human response data. It can be seen that the size measurement of a 3D microtumor quite faithfully reflects the clinical characterization of tumors in vivo. In vivo data taken from Lee et al. 2005.

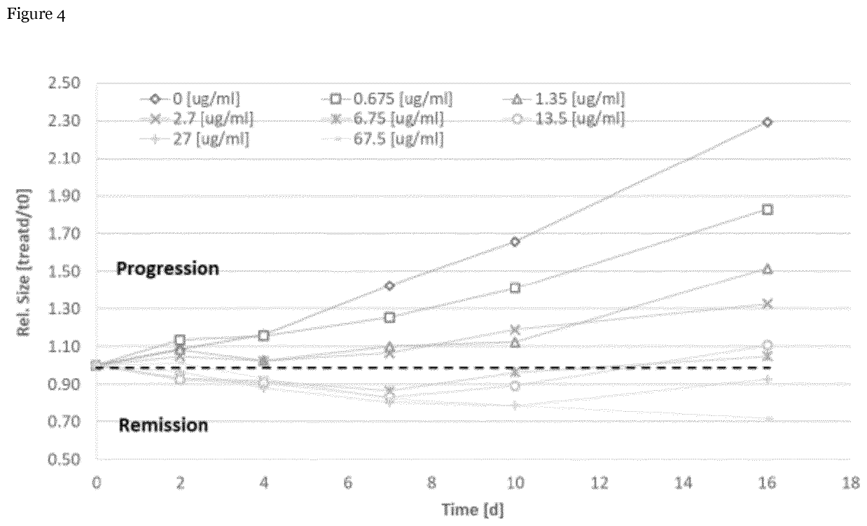

FIG. 4 shows an example of a dose to growth correlation study with Irinotecan at different concentrations, to determine which concentration range to be used for combinatorial drug testing. To assess whether the assay causes concentration-dependent effects on the growth of tumor microtissues, pancreatic microtumors were used and treated with indicated concentrations of Irinotecan. In brief, pancreatic microtumors were produced from the pancreatic cancer cell line Panc-1 (ATCC® CRL-1469™) in co-culture with the mouse fibroblast cell line NIH3T3 (ATCC® CRL-1658™). Both cells were expanded in cell culture flasks using Dulbecco's modification of Eagle medium (DMEM) supplemented with 10% fetal calf serum. After reaching confluency the were removed from the cell culture flask enzymatic digestion (Trypsin). Cell numbers of both cell types were assessed using a Neubauer chamber, and the respective cell number mixed to produce the co-culture microtumors. Seventy ul of the cell mixture was inoculated in each well of a non-adhesive 96-well plates (GravityTRAP™) using Dulbecco's modification of Eagle medium (DMEM) supplemented with 10% horse serum. Pancreatic microtumors were matured in a mammalian cell culture incubator at 37° C. with 5% CO₂. Four days after the cells formed pancreatic tumor microtissues they were dosed with Irinotecan at indicated concentrations adapted according to clinical-used dosages. After 48 hours the drug was removed by changing the culture medium with drug-free medium. The size of the pancreatic microtumors was monitored prior dosing and continuously after dosing using bright field microscopy. Growth is presented relative to the size of the pancreatic tumor microtissue prior compound treatment t₀ (adapted from the RECIST criteria). A clear correlation of dose to growth can be observed.

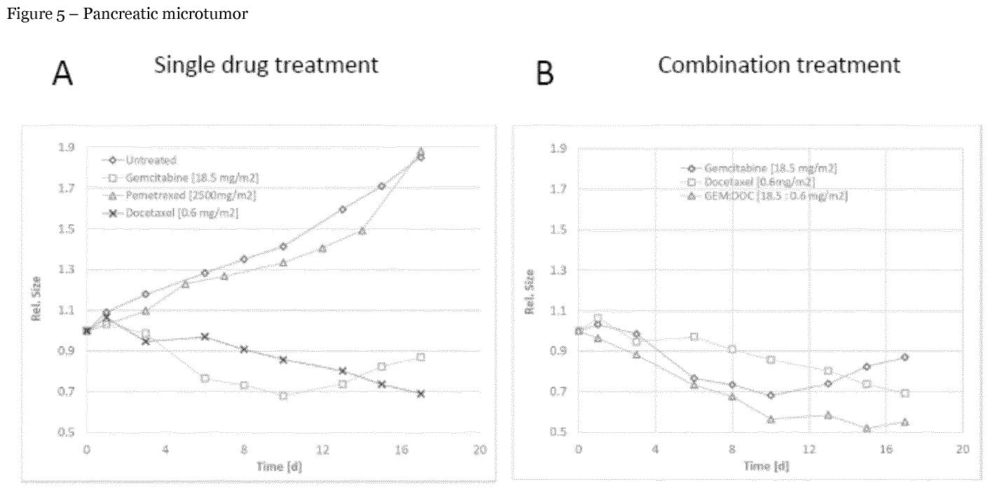

FIG. 5 shows single and combinatorial effects applying pancreatic microtumors (production described in FIG. 4). All drugs used were dissolved according to the manufactures protocol. Gemcitabine (approved against PC), Docetaxel (approved against PC) and Pemetrexed (not approved), inhibiting DNA and RNA synthesis, were dosed for 8h with a 48h gap (FIG. 5A). In contrast to the two approved drugs Gemcitabine and Docetaxel, Pemetrexed did not affect tumor growth demonstrating the specificity of the method. Whereas Gemcitabine unfolds its effect quickly, the response to Docetaxel was much slower. However, after day 10 the microtumors treated with Gemcitabine relapsed whereas Docetaxel treatment was more sustainable (FIG. 5A). Combining the two drugs lead to a fast and sustainable response (FIG. 5B).

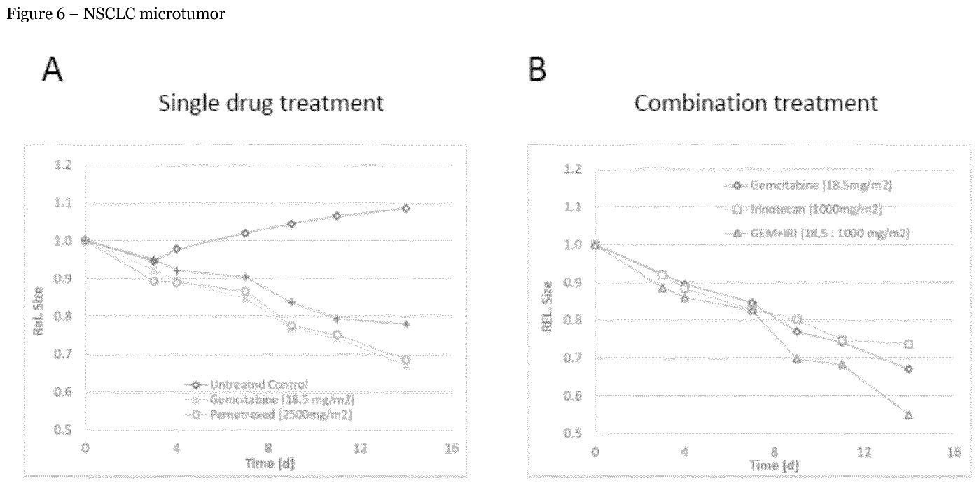

FIG. 6 shows single and combinations treatment applying non-small cell lung cancer (NSCLC) model. The NSCLC model was produced by mixing A549 NSCLC cells (ATCC® CCL-18S™) with human lung fibroblasts (Wi38, ATCC® CCL-7S™) in a non-adhesive 96-well plate (GravityTRAP™). Drugs were dosed 2× for 8h with 48h gap. While Pemetrexed was not active against pancreatic microtumors we observed tumor remission of NSCLC microtumors (FIG. 6A). A combinatorial effect was observed combing Gemcitabine and Irinotecan resulting in slightly higher efficiency as compared to the single drug doses.

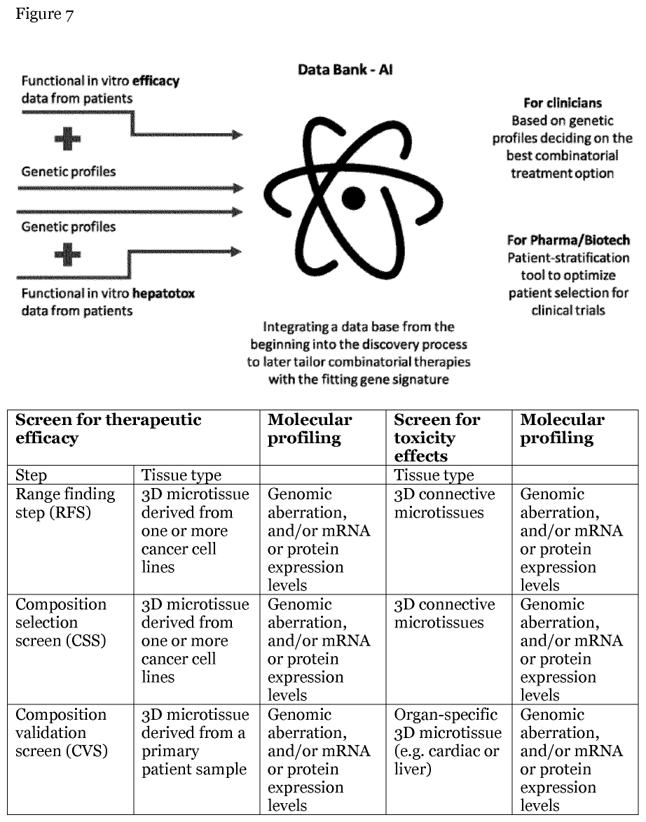

FIG. 7 shows basic steps of the method according to the invention, and optional embodiments (shown in italics). The entire package allows the generation of a data bank comprising individual data sets based on 3D method-specific functional data for efficacy and toxicity of given drug combinations with the corresponding genetic profiles of
3D microtissues derived from one or more cancer cell lines,
3D microtissues derived from primary patient sample,
3D microtissues representing connective tissue, and/or
tissue specific microtissues.

FIG. 8: Key for efficient combinatorial drug discovery is to maintain high throughput capabilities without losing biological relevance. FIG. 8 exemplifies the hit selection and lead validation stage with the associated decreasing number of data points of each stage. “B” refers to step b) in claim 1 (CSS), while “C” refers to step c) in claim 1 (CVS).

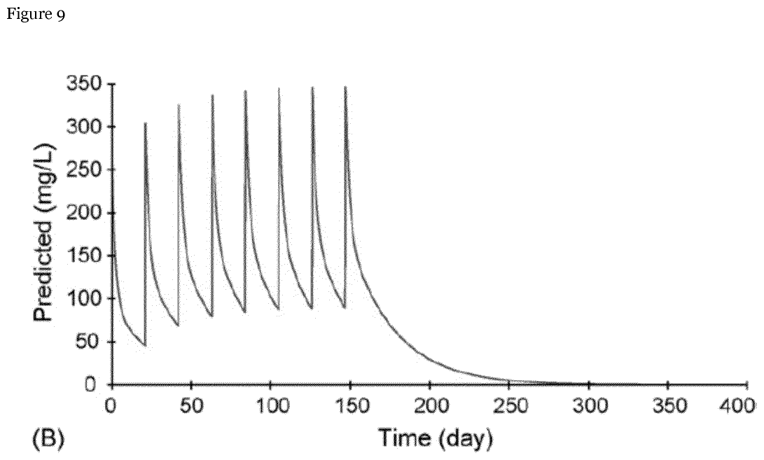

FIG. 9 shows the drug serum titer as a function of time following eight infusions every 21 days. Between the different administrations, the serum titer quickly decreases again. Due to the clearance of drugs in the body the tumor tissue retention time is mostly in the range of hours. Figure taken from Cartron et al, 2007.

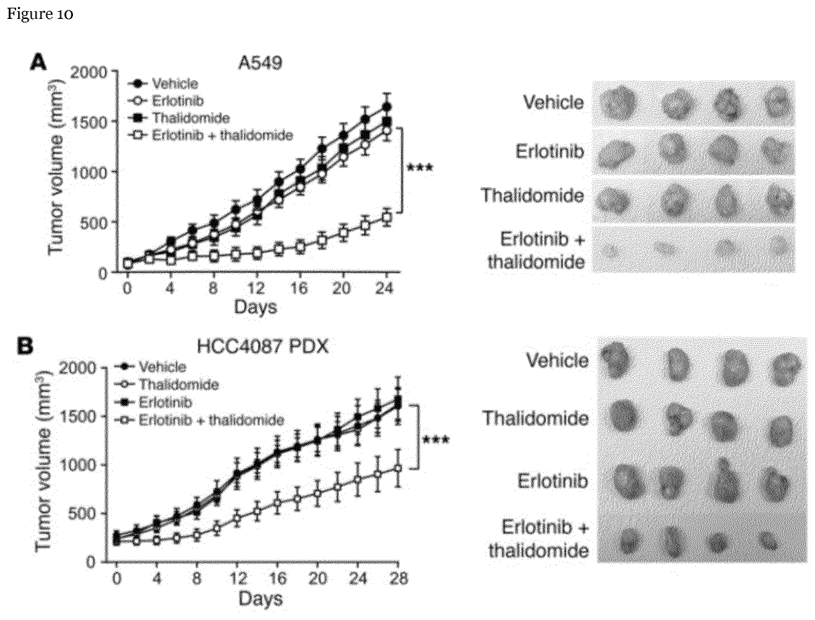

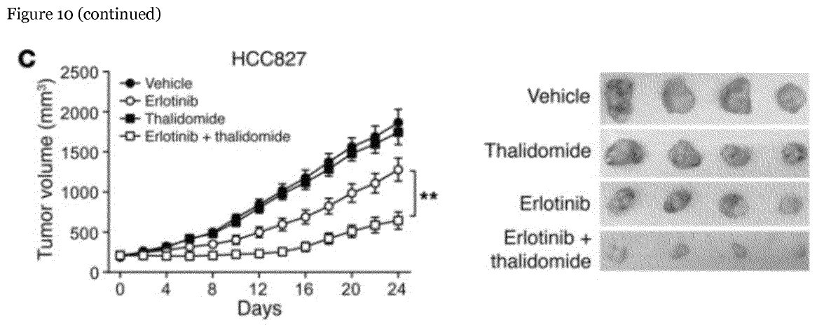

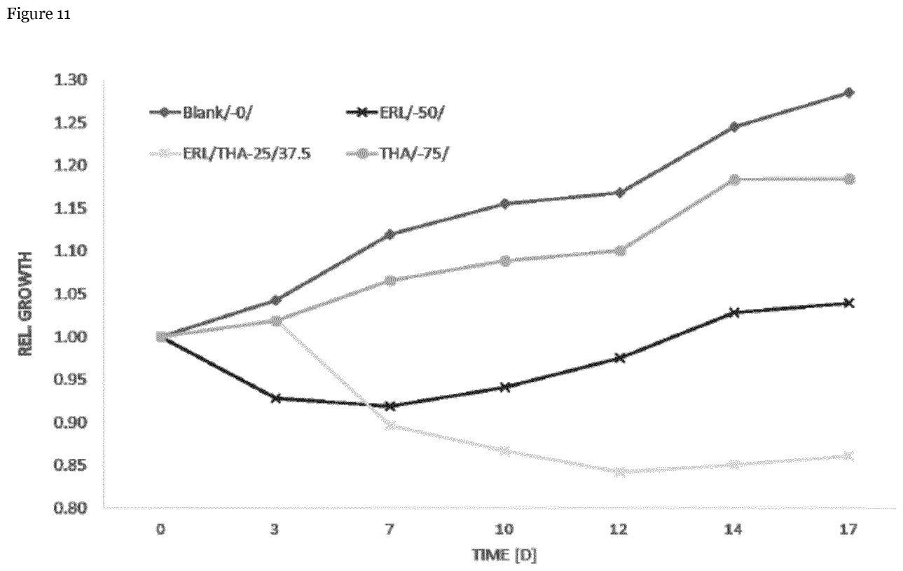

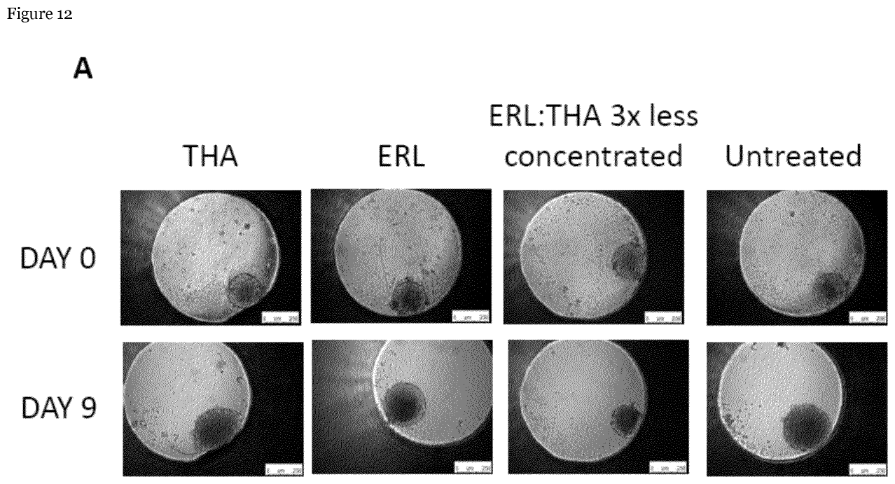

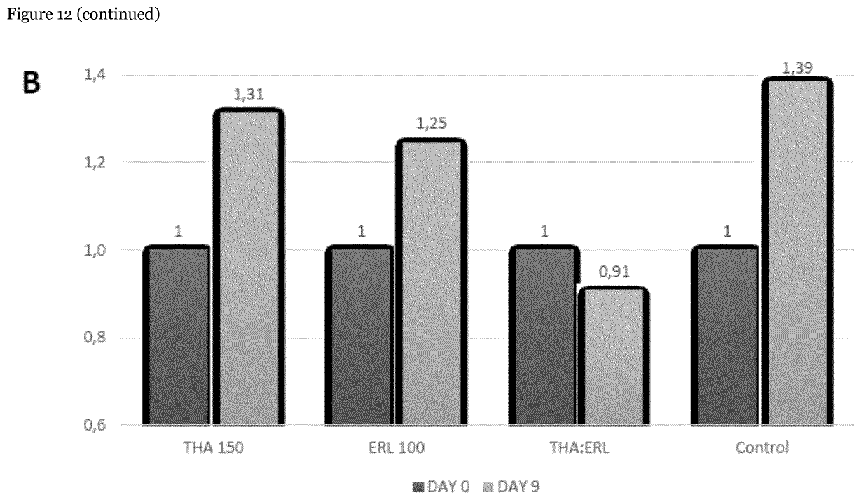

FIG. 10 demonstrates combinatorial drug testing in mice using xenografts from two different non-small cell lung cancer (NSCLC) cell lines (A549 and FICC827) and a patient-derived xenograft (HCC4087). The data demonstrates that single treatments of Erlotinib (approved against NSCLC), a tyrosine kinase inhibitor, and Thalidomide (not approved), an immunomodulatory drug, in the mouse model have a clear synergistic effect. The work done by Gong and coworkers was the basis to evaluate whether the 3D in vitro test method can recapitulate the in vivo results [Gong et al. 2018] shown in FIGS. 11 and 12.

FIG. 11 demonstrates single and combination treatment using the cell line-based non-small cell lung cancer model (A549, Wi38) as described in FIG. 6. Single and drug combinations were dosed 2× for 8h with a gap of 48h and growth monitored over time. Drug concentrations were adapted from Gong et al. As shown already by Gong et al. in vivo Thalidomide has no significant impact on tumor growth remission, whereas Erlotinib leads to reduced growth but growth relapses after 7 days. Applying 2× less concentrated drugs in the combination a clear superior response was observed over time confirming pre-clinical animal-based results published by Gong et al.

FIG. 12 exemplifies that a similar outcome as shown in FIGS. 10 and 11 can be achieved with non-small cell cancer (NSCLC) cells directly derived from patients. A NSCLC resection was dissociated, and the cell Suspension used to produce microtumors in a multi-well format (FIG. 12A). Drugs were dosed 2× for 8h with a 48h gap. Whereas Erlotinib and Thalidomide single doses had hardly an impact on tumor growth a clear response was reached with both drugs in combination 3× less concentrated. FIG. 12B displays quantitative values of the relative sizes of day 0 and 9, normalized to day 0 according to the RECIST criteria.

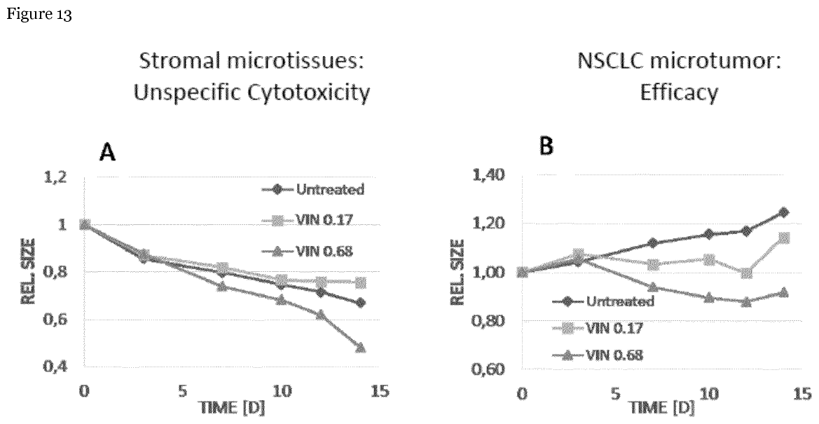

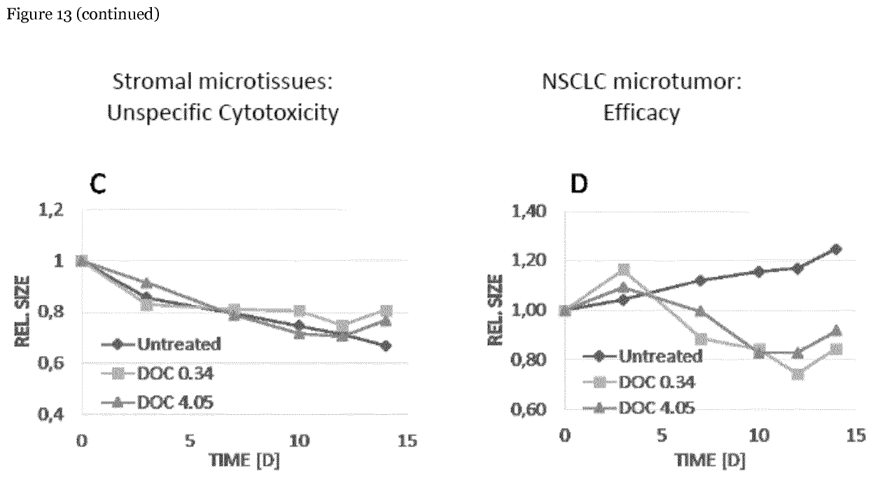

FIG. 13 exemplifies tox testing of single drugs using connective/stromal microtissues (Wi38) and efficacy testing on non-small cell tumor microtissues (A549; Wi38). Vinorelbin and Docetaxel, both disrupting microtubule, were dosed 2× for 8h with an 8h gap. As compared to the untreated control of the stromal microtissues high dose of Vinorelbin (0.68 ug/ml) displayed elevated cytotoxicity in contrast to Docetaxel (4.05 ug/ml) (FIGS. 13A and C). Comparing efficacy Vinorelbin in high concentration result in less impact on tumor growth compared to low and high concentrated Docetaxel (FIGS. 13B and D). In the framework of compound classification Docetaxel would be favored due to less toxicity and higher efficacy for further development.

### EXAMPLES

The present invention allows for a harmonized analysis starting from a screening to a test using patient material. This makes it possible to align the screening data retrospectively with patient data. In the following, two examples are summarized in tables 6 and 7.

TABLE 4
RECIST criteria to evaluate therapy outcome
(Eisenhower et al. 2009 European Journal of Cancer)

| | |
|---|---|
| Complete | Disappearance of all target lesions. Any pathological lymph nodes |
| Response | (whether target or non-target) must have reduction in short axis to <10 |
| (CR) | mm. |
| Partial | At least a 30% decrease in the sum of diameters of target lesions, |
| Response (PR) | taking as reference the baseline sum diameters. |
| Progressive | At least a 20% increase in the sum of diameters of target lesions, |
| Disease (PD) | taking as reference the smallest sum on study (this includes the |
| | baseline sum if that is the smallest on study). In addition to the relative |
| | increase of 20%, the sum must also demonstrate an absolute increase |
| | of at least 5 mm. (Note: the appearance of one or more new lesions is |
| | also considered progression). |
| Stable Disease | Neither sufficient shrinkage to qualify for PR nor sufficient increase to |
| (SD) | qualify for PD, taking as reference the smallest sum diameters while |
| | on study. |
| Relapse-free | Length of time after primary treatment for a cancer ends that the |
| survival (RFS) | patient survives without any signs or symptoms of that cancer |

TABLE 5
Clinically-adapted test-based criteria to evaluate therapy success

| | |
|---|---|
| Remission | ≥20% decrease in microtumor size, |
| (RE) | Reference: Size t₀ |
| Progression | ≥20%-100% increase in microtumor size |
| (PR) | Reference: Untreated Control and t₀ |
| Stable | +/−20% increase/decrease in size |
| Response | Reference: t₀ |
| (SR) | |
| Time to | Time to re-entry into the growth phase |
| Relapse | Reference: Size eₘₐₓ |
| (TR)* | |
| Maximal | Maximum increase in size |
| response | Reference: Size t₀ |
| (MR)* | |
| Time to MR | Time until MR is reached |
| (TMR)* | Reference: Size MR |
| Potency | Lowest concentration to reach MR relative |
| (PO)* | to cmax (c_{MR}/cₘₐₓ) |
| | Reference: cₘₐₓ of the respective drug |
| ΔT/ΔC | Ratio of treatment effect (ΔT) to untreated control (ΔC) |

Time window: 14 days
Concentration-dependent outcome analysis
*Indicates criteria to indicate effective therapies

TABLE 6
Case example 1
Pancreatic cancer treated with Gemcitabine and Irinotecan

| | Pancreatic Cancer (Panc-1) | | | | | |
|---|---|---|---|---|---|---|
| Indication | Gemcitabine | | | Irinotecan | | |
| Drug | (median cₘₐₓ = 20 ug/ml) | | | (median cₘₐₓ = 5 ug/ml) | | |
| Potency | 0.675 | | | 13.5 | | |
| (PO) [c_{MR}/cₘₐₓ] | | | | | | |
| Concentration | 0.1 | 0.5 | 13.5 | 0.675 | 6.75 | 67.5 |
| [ug/ml] | | | | | | |
| Response | SR | SR | RE | PR | SR | RE |
| Time until | 10 | 10 | — | — | 7 | — |
| relapse | | | | | | |
| (TR) [d] | | | | | | |
| Maximal | 0.82 | 0.68 | 0.56 | — | 0.86 | 0.72 |
| response | | | | | | |
| (MR) [%] | | | | | | |
| Time to | 10 | 10 | 14 | — | 7 | 14 |
| MR (TMR) | | | | | | |
| [d] | | | | | | |
| ΔT₁₄/ΔC₁₄ | −0.25 | −0.23 | −0.68 | 1.04 | 0.06 | −0.36 |

TABLE 7
Case example II
Lung cancer treated with Gemcitabine and Irinotecan and a combination thereof

| | Lung Cancer (A549) | | | | | | | | | | | |
|---|---|---|---|---|---|---|---|---|---|---|---|---|
| Indication | Gemcitabine | | | Irinotecan | | | | | | | | |
| Drug | (median cₘₐₓ = 20 ug/ml) | | | (median cₘₐₓ = 5 ug/ml) | | | Gemcitabine:Irinotecan | | | | | |
| Potency | 0.015 | | | 1.35 | | | 0.025:1.35 | | | | | |
| (PO) [c_{MR}/cₘₐₓ] | | | | | | | | | | | | |
| Concentration | 0.1 | 0.3 | 0.5 | 6.75 | 13.5 | 27 | 0.5:27.0 | 0.5:6.75 | 0.1:27.0 | 0.1:6.75 | 0.3:27.0 | 0.3:13.5 |
| [ug/ml] | | | | | | | | | | | | |
| Response | RE | RE | RE | ST | RE | RE | RE | RE | RE | RE | RE | RE |
| Time until | — | — | — | 5 | 7 | — | — | — | — | — | — | — |
| relapse | | | | | | | | | | | | |
| (TR) [d] | | | | | | | | | | | | |
| Maximal | 0.77 | 0.66 | 0.67 | 0.88 | 0.93 | 0.74 | 0.55 | 0.70 | 0.69 | 0.71 | 0.66 | 0.65 |
| response | | | | | | | | | | | | |
| (MR) [%] | | | | | | | | | | | | |
| Time to | 14 | 14 | 14 | 4 | 7 | 14 | 14 | 14 | 9 | 11 | 14 | 14 |
| MR (TMR) | | | | | | | | | | | | |
| [d] | | | | | | | | | | | | |
| ΔT₁₄/ΔC₁₄ | −2.72 | −4.04 | −3.87 | −0.60 | −0.32 | −3.09 | −5.29 | −3.49 | −3.62 | −3.45 | −4.03 | −4.13 |

### REFERENCES

Cartron G et al., Crit Rev Oncol Hematol. 2007 April; 62(1):43-52. Epub 2007 Feb. 6.
Lee T K et al., Carcinogenesis. 2004 December; 25(12):2397-405. Epub 2004 Aug. 5.
Gong et al., The Journal of Clinical Investigation 2018 June, 128(6)
Kelm et al., J Biotechnol. 2005 Aug. 4; 118(2):213-29
Tuschl et al., Chem Biol Interact. 2009 Sep. 14; 181 (1): 124-37
Bokhari et al., J Anat. 2007 October; 211(4):567-76
Beckwitt et al., Exp Cell Res. 2018 Feb. 1; 363(1): 15-25
Proctor et al., D. Arch Toxicol. 2017 August; 91(8):2849-2863
Sidorov et al., Acta Biomater. 2017 Jan. 15; 48:68-78
Giacomelli et al., Development. 2017 Mar. 15; 144(6): 1008-1017
Hansen et al., T. Circ Res. 2010 Jul. 9; 107(1):35-44
Kijanska & Kelm J. In vitro 3D Spheroids and Microtissues: ATP-based Cell Viability and Toxicity Assays. Assay Guidance Manual [Internet]. Bethesda (Md.): Eli Lilly & Company and the National Center for Advancing Translational Sciences; 2004-2016 Jan. 21
Kelm et al, Drug Discov Today. 2018 Jul. 30. pii: 51359-6446(18)30225

1. A method for characterizing a physiological effect of a composition comprising two or more active drug compounds, the method comprising the steps of:
b) a composition selection screen (CSS), in which screen a 3D microtissue derived from one or more cell lines is exposed to said composition comprising two or more active drug compounds and/or
c) a composition validation screen (CVS), in which screen 3D microtissue derived from a primary patient sample is exposed to the composition of step b),
so as to characterize a physiological effect of said composition on the 3D microtissue.

2. The method according to claim 1, wherein at least one parameter representing the characterized physiological effect is generated or determined in the method.

3. The method according to claim 1, which method further comprises:
a) a range finding step (RFS), in which step a plurality of 3D microtissues derived from one or more cell lines are exposed to different concentrations of each compound of the composition, so as to determine suitable concentration ranges of the compounds.

4. The method according to claim 1, which method further comprises a step of obtaining a molecular profile of at least one of
the 3D microtissues derived from one or more cell lines, and/or
the 3D microtissues derived from a primary patient sample.

5. The method according to claim 1, wherein a step of molecular profiling is used to detect genomic aberrations, and/or mRNA or protein expression levels.

6. The method according to claim 1, in which method the parameter representing the characterized physiological effect is determined over time in at least one of step a), b) and/or c).

7. The method according to claim 1, in which method the parameter representing the characterized physiological effect is the size of the 3D microtissue.

8. The method according to claim 1, in which method actual size, relative size and/or relative size change over time is determined in at least one of step a), b) and/or c).

9. The method according to claim 7, in which method the size determination of the 3D microtissue refers to at least one parameter selected from the group consisting of:
diameter
perimeter
volume, and
area of an optical cross section.

10. The method according to claim 8, in which method the size is determined in at least one of step a), b) and/or c) over a period of >1 and <20 days.

11. The method according to claim 1, in which method the 3D microtissue is exposed to the active drug compound (step a) or to the composition comprising two or more active drug compounds (steps b and c) only once, by application of a defined bolus.

12. The method according to claim 1, in which method the 3D microtissue is exposed to the active drug compound (step a) or to the composition comprising two or more active drug compounds (steps b and c) two or more times, by application of a defined bolus each.

13. The method according to claim 1, wherein the composition comprising two or more active drug compounds is removed after the microtissue was exposed thereto for a given period of time.

14. The method according to claim 1, wherein further a step of composition toxicity testing is provided, in which step:
(i) a microtissue representing connective tissue is exposed to the composition of step b),
and/or
(ii) a tissue specific microtissue is exposed to the composition of step c), so as to characterize a physiological effect of said composition on said microtissue.

15. The method according to claim 1, wherein at least one 3D microtissue has been produced in a hanging drop culture system or a low adhesion well culture system.

16. The method according to claim 1, wherein the molecular profile of at least one 3D microtissue is correlated with at least one parameter which characterizes the physiological effect as obtained in the composition selection screen (CSS) or the composition validation screen (CVS) of said 3D microtissue.

17. The method according to claim 16, which method further comprises the step of creating or feeding a database with datasets comprising at least the following entries each:
a) at least one molecular profile of at least one 3D microtissue, and
b) at least one parameter which characterizes the physiological effect as obtained in the composition selection screen (CSS) or the composition validation screen (CVS) of said 3D microtissue.

18. A method of screening a plurality of compositions comprising two or more active drug compounds, preferably from one or more libraries, which method comprises:
(i) the application of two or more methods as set forth in any of the aforementioned claims, with a different composition of two or more active drug compounds in each individual method, and/or
(ii) several pairs of steps b), c) and optionally a).

19. The method according to claim 18, wherein the compositions comprising two or more active drug compounds differ from one another by
a) the composition of active drug compounds, or
b) the dosages or concentrations of the active drug compounds in the composition.

20. The method according to claim 1, which method further comprises at least one Step selected from the group consisting of:
a) synthesizing the active drug compounds that are in the compositions,
b) composing the compositions comprising two or more active drug compounds, and/or
c) creating a library comprising active drug compounds that are comprised in the compositions and/or compositions comprising two or more active drug compounds.

21. A method of creating a database, in which the molecular profile of at least one 3D microtissue is correlated with the result of a composition selection screen (CSS) or a composition validation screen (CVS) of said 3D microtissue.

22. The method according to claim 21, wherein the molecular profile of at least one 3D microtissue is correlated with at least one parameter which characterizes the physiological effect as obtained in the composition selection screen (CSS) or the composition validation screen (CVS) of said 3D microtissue.
